(19) United States
(12) Patent Application Publication
(10) Pub. No.: US 2023/0405169 A1
(43) Pub. Date: Dec. 21, 2023

(54) Helmet Cleaning, Sterilization, Deodorization and Drying System and Method

(71) Applicant: LIFIKEYS TECHNOLOGY CORPORATION, Taipei City (TW)

(72) Inventor: Hao-Yu Cheng, Taipei City (TW)

(21) Appl. No.: 18/325,187

(22) Filed: May 30, 2023

(30) Foreign Application Priority Data
Jun. 16, 2022 (TW) 111122479
Mar. 17, 2023 (TW) 112110121

(51) Int. Cl. A61L 2/22; B08B 5/02; B08B 7/02; B08B 3/08; B08B 7/04; B08B 13/00; A61L 2/20; A61L 2/26

(52) U.S. Cl. CPC A61L 2/22; A61L 2202/26; B08B 7/02; B08B 3/08; B08B 7/04; B08B 13/00; A61L 2/202; A61L 2/26; A61L 2202/17; A61L 2202/11; A61L 2202/122; A61L 2202/123; A61L 2202/15; A61L 2202/14; B08B 5/02

(57) ABSTRACT

A helmet cleaning, sterilization, deodorization and drying system includes a main body, an ultraviolet lamp module, a fog generator, a warm air generator, a first exhaust module, a controlling system module, a helmet cleaning and sterilization compartment, a containing space, a cleaning liquid tank and a gas conduit. The controlling system module is electrically connected to the ultraviolet lamp module, the fog generator, the warm air generator and the first exhaust module. The helmet sterilization cleaning compartment is located in the main body and includes a storage space, a plate and a ventilation hole. The storage space is used for placing the helmet. The plate is located on the bottom of the storage space. The ventilation hole is located on the plate. The containing space is located in the main body. The cleaning liquid tank is used for containing the antibacterial fog cleaning liquid.

### CROSS REFERENCE TO RELATED APPLICATIONS

This application claims priority of Application No. 111122479 filed in Taiwan on Jun. 16, 2022 under 35 U.S.C. § 119, and priority of Application No. 112110121 filed in Taiwan on Mar. 17, 2023 under 35 U.S.C. § 119 the entire contents of all of which are hereby incorporated by reference.

### BACKGROUND

### 1. Technical Field

The present invention relates to a helmet cleaning, sterilization, deodorization and drying system and method; more particularly, the present invention relates to a helmet cleaning, sterilization, deodorization and drying system and method which can quickly gaseous fume dry-clean from all possible angles, clean, sterilize, deodorize and dry the lining of a helmet.

### 2. Description of the Related Art

To ensure the safety of motorcycle riders, the law requires each motorcycle rider to wear a helmet. Since the inside of the helmet is made of polystyrene and covered with a layer of cotton, the sweat of the rider or the rainwater will be absorbed by the cotton layer. Over time, bacteria and mold will grow on the cotton layer, emitting a foul odor and even causing skin problems or hair loss on the rider's scalp.

In order to clean the inside of the helmet, Patent No. M564453, titled “Safety helmet sterilization machine,” uses the technology of spraying medicated mist through ultrasonic vibration. The mist particles can clean, disinfect, and sterilize the inside of the helmet. The ultrasonic vibration from the patent generates mist particles ranging from 6 to 100 microns in size, which are large and contain water, but the machine does not have a drying function. Therefore, the helmet lining may still contain moisture, leading to concerns about the growth of bacteria and mold.

Therefore, it is worthwhile to explore how to provide a device that can quickly clean, disinfect, deodorize, and dry the helmet liner to keep it clean and fresh and prevent the user from developing scalp problems after wearing it.

### SUMMARY

The object of the present invention is to provide a helmet cleaning, sterilization, deodorization and drying system which can quickly gaseous fume dry-clean from all possible angles, clean, sterilize, deodorize and dry the lining of the helmet.

To achieve the abovementioned object, a helmet cleaning, sterilization, deodorization and drying system of the present invention includes a main body, an ultraviolet lamp module, a fog generator, a warm air generator, a first exhaust module, a controlling system module, a helmet cleaning and sterilization compartment, a containing space, a cleaning liquid tank and a gas conduit. The controlling system module is electrically connected to the ultraviolet lamp module, the fog generator, the warm air generator and the first exhaust module. The helmet cleaning and sterilization compartment is located in the main body and includes a storage space, a plate and at least one ventilation hole. The storage space is used for placing the helmet. The plate is located on the bottom of the storage space. The ventilation hole is located in the plate. The containing space is located in the main body. The cleaning liquid tank is used for containing the antibacterial fog cleaning liquid. The gas conduit is located in the containing space and linked to the helmet cleaning and sterilization compartment. Via the abovementioned structure, the antibacterial fog cleaning liquid forms a cleaning, sterilizing and deodorizing fog via the fog generator, and the cleaning, sterilizing and deodorizing fog enters the helmet cleaning and sterilization compartment via the gas conduit and the ventilation hole. The warm air generator is used for heating the air such that the first exhaust modules produces warm air, and the warm air enters the helmet cleaning and sterilization compartment via the gas conduit and the ventilation hole. The fog molecules generated by heating are gas molecules that are smaller than the water mist sprayed by conventional ultrasonic vibration, and the fog particles contain no water.

According to one embodiment of the present invention, the helmet cleaning, sterilization, deodorization and drying system further includes an ozone generator, a second exhaust module, at least one fragrance module, a first turbo fan module, a second turbo fan module, a humidity sensor, an odor sensor and a vibration module.

### BRIEF DESCRIPTION OF THE DRAWINGS

FIG. 1 illustrates a schematic drawing of the helmet cleaning, sterilization, deodorization and drying system in one embodiment of the present invention.

FIG. 2 illustrates a sectional view of the helmet cleaning, sterilization, deodorization and drying system in one embodiment of the present invention.

FIG. 3 illustrates a schematic drawing of a helmet in the helmet cleaning, sterilization, deodorization and drying system in one embodiment of the present invention.

FIG. 4 illustrates a system structure drawing of the helmet cleaning, sterilization, deodorization and drying system in one embodiment of the present invention.

FIG. 5 illustrates a step flowchart of the helmet cleaning, sterilization, deodorization and drying method in one embodiment of the present invention.

FIG. 6 is a schematic drawing illustrating how the antibacterial fog cleaning liquid becomes the cleaning, sterilizing and deodorizing fog in one embodiment of the present invention.

FIG. 7 is a schematic drawing illustrating how the air becomes warm air in one embodiment of the present invention.

### DETAILED DESCRIPTION OF THE EMBODIMENTS

In order to further describe the technical content of the present invention, a preferred specific embodiment is given as following description.

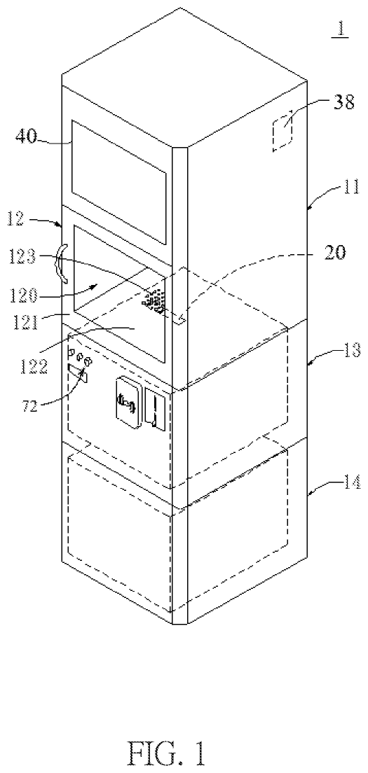

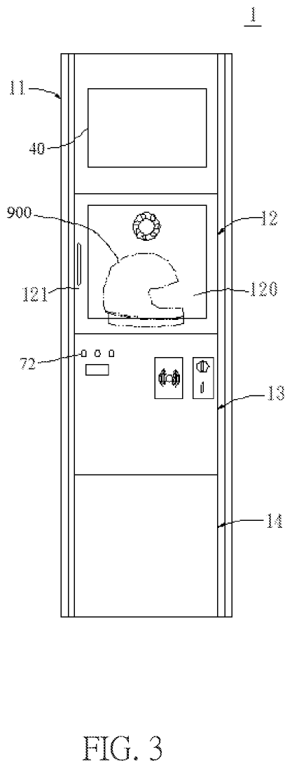

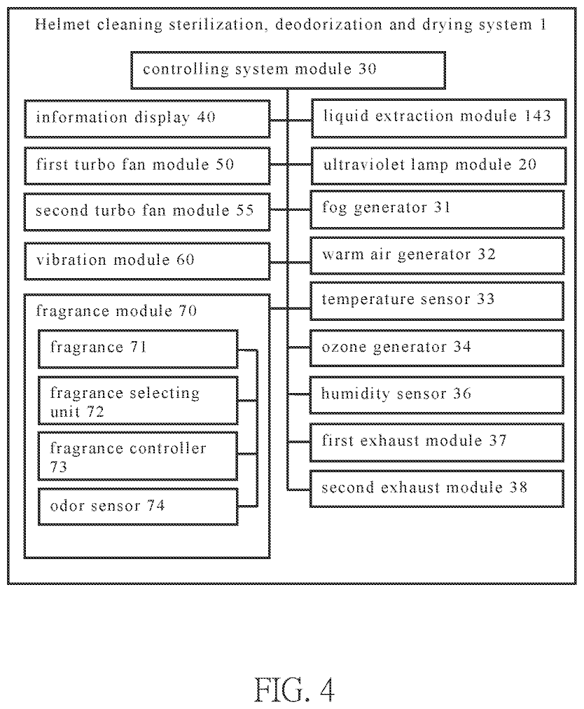

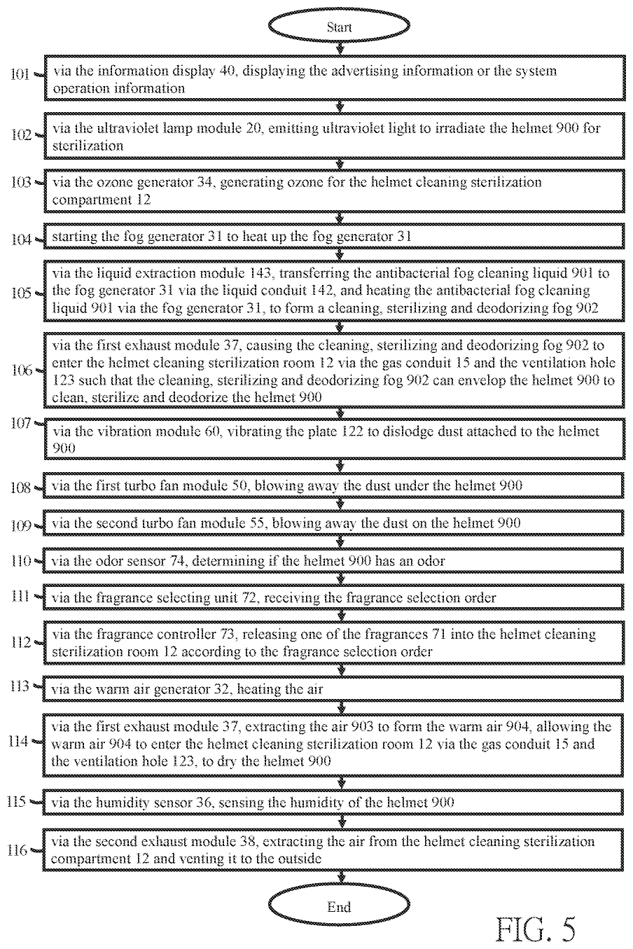

Please refer to FIG. 1 to FIG. 7 about the helmet cleaning, sterilization, deodorization and drying system and the helmet cleaning, sterilization, deodorization and drying method in one embodiment of the present invention.

As shown in FIG. 1 to FIG. 4 and FIG. 6, in one embodiment of the present invention, the helmet cleaning, sterilization, deodorization and drying system 1 and the helmet cleaning, sterilization, deodorization and drying method can quickly clean, sterilize, deodorize and dry the lining of the helmet 900 via an antibacterial fog cleaning liquid 901. The composition of the antibacterial fog cleaning liquid 901 includes water, a smoking agent and an antibacterial deodorant detergent. After being heated, the antibacterial fog cleaning liquid 901 becomes the cleaning, sterilizing and deodorizing fog 902 with the cleaning and disinfecting function. The helmet cleaning, sterilization, deodorization and drying system 1 includes a main body 11, a helmet cleaning and sterilization compartment 12, a containing space 18, a cleaning liquid tank 141, a liquid conduit 142, a liquid extraction module 143, a gas conduit 15, an ultraviolet lamp module 20, a controlling system module 30, a fog generator 31, a warm air generator 32, a temperature sensor 33, an ozone generator 34, a humidity sensor 36, a first exhaust module 37, a second exhaust module 38, an information display 40, a first turbo fan module 50, a second turbo fan module 55, a vibration module 60 and a fragrance module 70.

The main body 11 is a cuboid machine. The helmet cleaning and sterilization compartment 12 is located in the main body 11 and includes a storage space 120, a door body 121, a plate 122 and a plurality of ventilation holes 123. The storage space 120 is used for placing the helmet 900. The door body 121 is an openable door and used for covering the storage space 120. The plate 122 is located on the bottom of the storage space 120. The plurality of ventilation holes 123 are located on the plate 122 for allowing the air to pass through, but the amount of the ventilation holes 123 can be changed to be at least one according to design requirements.

The containing space 18 is located in the main body 11 and includes a first space 13 and a second space 14, and the second space 14 is located under the first space 13. The cleaning liquid tank 141 is located in the second space 14 and used for containing the antibacterial fog cleaning liquid 901. Two ends of the liquid conduit 142 are respectively linked to the cleaning liquid tank 141 and the liquid extraction module 143.

The gas conduit 15 is a ventilation tube and is located in the containing space 18 and linked to the helmet cleaning and sterilization compartment 12 for allowing the air to flow to the helmet cleaning and sterilization compartment 12. The ultraviolet lamp module 20 is located under the plate 122, and the ultraviolet light emitted by the ultraviolet lamp module 20 can pass through the ventilation hole 123 to irradiate the helmet 900 on the plate 122 for sterilization; however, the position of the ultraviolet lamp module 20 is not limited to the abovementioned description, for it can also be located at the top of the storage space 120.

The controlling system module 30 is a central processor unit, which is electrically connected to the ultraviolet lamp module 20, the liquid extraction module 143, the fog generator 31, the warm air generator 32, the temperature sensor 33, the ozone generator 34, the humidity sensor 36, the first exhaust module 37, the second exhaust module 38, the information display 40, the first turbo fan module 50, the second turbo fan module 55, the vibration module 60 and the fragrance module 70, and is used for controlling the functions of those modules. The controlling system module 30 is located in the containing space 18. The fog generator 31 is linked to the liquid extraction module 143, and the fog generator 31 is used for heating the antibacterial fog cleaning liquid 901 extracted by the liquid extraction module 143 to form the cleaning, sterilizing and deodorizing fog 902. The warm air generator 32 is used for heating the air 903. The temperature sensor 33 is used for sensing the temperature of the air 903 and each of the units in the first space 13. The ozone generator 34 is linked to the helmet cleaning and sterilization compartment 12 and used for producing ozone for the helmet cleaning and sterilization compartment 12. The humidity sensor 36 is located in the helmet cleaning and sterilization compartment 12 and is used for sensing the humidity of the helmet 900. The first exhaust module 37 is an exhaust fan located under the ventilation hole 123 and is used for propelling air into the helmet cleaning and sterilization compartment 12. The second exhaust module 38 is an exhaust fan located at the top of the helmet cleaning and sterilization compartment 12 and is used for extracting the air from the helmet cleaning and sterilization compartment 12 and venting it to the outside.

The information display 40 is a screen, is located on the main body 11, and is used for displaying the advertising information or system operating information. The first turbo fan module 50 is located under the plate 122 and includes a conduit 51, and the conduit 51 is linked to the ventilation hole 123. The first turbo fan module 50 is used for propelling the airflow through the ventilation hole 123 to blow away dust under the helmet 900. The second turbo fan module 55 is located at the top of the helmet cleaning and sterilization compartment 12 and is used for propelling the airflow to blow away dust on the helmet 900. The current generated by the turbo fan module should have a flow rate at least 1 m³/min to 10 m³/min and a current pressure of at least 500 pa to 3000 pa; the turbo fan module should have a rotational speed of at least 1100 RPM to 5000 RPM or more or generate a strong current having a speed of at least 10 CFM or more. The vibration module 60 is connected to the plate 122 and is used for vibrating the plate 122 to dislodge dust attached to the helmet 900.

The fragrance module 70 is used for producing an aroma. The fragrance module 70 is used for allowing a user to select the aroma and includes a plurality of fragrances 71, a fragrancy selecting unit 72, a fragrancy controller 73 and an odor sensor 74. The plurality of fragrances 71, for example, are a plurality of sprayers which contain essences, essential oils or perfumes, and the plurality of fragrances 71 respectively provide different aromas, such as floral or fruity fragrances. The fragrancy selecting unit 72 is a button located on the main body 11 and is used for allowing the user to select the user's favorite fragrance. The fragrancy controller 73, for example, is a processor chip which is electrically connected to the plurality of fragrances 71, the fragrancy selecting unit 72 and the odor sensor 74 to control the functions of those units. The odor sensor 74 is located at the top of the helmet cleaning and sterilization compartment 12 and is used to determine if the helmet 900 has an odor.

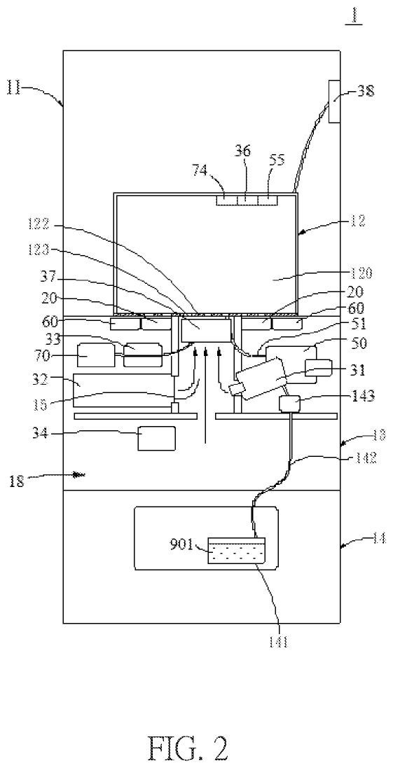

As shown in FIG. 2 to FIG. 5, the helmet cleaning, sterilization, deodorization and drying method can be programed as software and applied to the helmet cleaning, sterilization, deodorization and drying system 1. When the user wants to use the helmet cleaning, sterilization, deodorization and drying system 1 to clean the helmet 900, the helmet cleaning, sterilization, deodorization and drying system 1 executes step 101 of the helmet cleaning, sterilization, deodorization and drying method: via the information display 40, displaying advertising information or the system operating information.

The information display 40 displays an advertising information or a system operating information, and the user can watch the operating instructions shown on the information display 40 and follow the instructions to insert money into the coin slot on the main body 11 or use a mobile payment method, place the helmet in the storage space 120 on the plate 122, and close the door body 121.

Then the helmet cleaning, sterilization, deodorization and drying system 1 executes step 102 of the helmet cleaning, sterilization, deodorization and drying method: via the ultraviolet lamp module 20, irradiating the helmet 900 with ultraviolet light for sterilization.

The ultraviolet lamp module 20 emits ultraviolet light, which irradiates the helmet 900 on the plate 122 to sterilize the helmet 900.

Then the helmet cleaning, sterilization, deodorization and drying system 1 executes step 103 of the helmet cleaning, sterilization, deodorization and drying method: via the ozone generator 34, generating ozone for the helmet cleaning and sterilization compartment 12.

The ozone generator 34 is an ozonizer of the prior art, which can produce ozone, and the ozone is transferred to the helmet cleaning and sterilization compartment 12 via the gas conduit 15 by the first exhaust module 37 to sterilize the helmet 900.

Then the helmet cleaning, sterilization, deodorization and drying system 1 executes step 104 of the helmet cleaning, sterilization, deodorization and drying method: starting the fog generator 31 to heat up the fog generator 31.

The fog generator 31 is a heater, which can start and heat up to a specific temperature range (such as 105 degrees to 380 degrees) within a specific time range (such as 30 seconds to 3 minutes). The temperature sensor 33 is a thermometer, which can sense the temperature of the fog generator 31 to ensure that the temperature of the fog generator 31 can be used to atomize the antibacterial fog cleaning liquid 901 later without overheating.

Then the helmet cleaning, sterilization, deodorization and drying system 1 executes step 105 of the helmet cleaning, sterilization, deodorization and drying method: via the liquid extraction module 143, extracting the antibacterial fog cleaning liquid 901 and transferring it to the fog generator 31 via the liquid conduit 142 and heating the antibacterial fog cleaning liquid 901 via the fog generator 31 to form a cleaning, sterilizing and deodorizing fog 902.

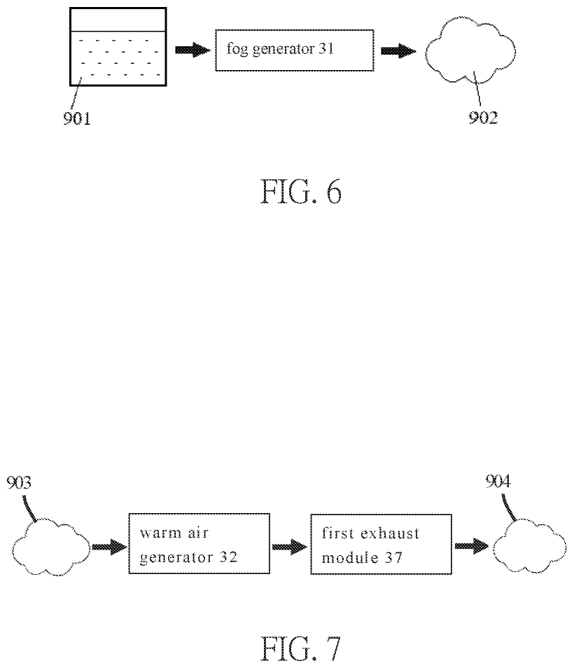

As shown in FIG. 2 and FIG. 6, the liquid extraction module 143 is a pump motor, which extracts the antibacterial fog cleaning liquid 901 from the cleaning liquid tank 141 for transferring to the fog generator 31 via the liquid conduit 142. The fog generator 31 heats up the antibacterial fog cleaning liquid 901 to form the cleaning, sterilizing and deodorizing fog 902 with the cleaning and sterilization function.

Then the helmet cleaning, sterilization, deodorization and drying system 1 executes step 106 of the helmet cleaning, sterilization, deodorization and drying method: via the first exhaust module 37, causing the cleaning, sterilizing and deodorizing fog 902 to enter the helmet cleaning and sterilization compartment 12 via the gas conduit 15 and the ventilation hole 123 such that the cleaning, sterilizing and deodorizing fog 902 envelops the helmet 900 to clean, sterilize and deodorize the helmet 900.

The first exhaust module 37 is an exhaust fan, which extracts the cleaning, sterilizing and deodorizing fog 902 via the gas conduit 15 and the ventilation hole 123 to cause the cleaning, sterilizing and deodorizing fog 902 to enter the helmet cleaning and sterilization compartment 12 to clean and sterilize the helmet 900.

Then the helmet cleaning, sterilization, deodorization and drying system 1 executes step 107 of the helmet cleaning, sterilization, deodorization and drying method: via the vibration module 60, vibrating the plate 122 to dislodge dust attached to the helmet 900.

The vibration module 60 is a vibration motor, which vibrates the plate 122 such that the dust attached to the helmet 900 which is on the plate 122 will be dislodged.

Then the helmet cleaning, sterilization, deodorization and drying system 1 executes step 108 of the helmet cleaning, sterilization, deodorization and drying method: via the first turbo fan module 50, blowing away dust under the helmet 900.

The first turbo fan module 50 is a fan, which can blow away any dust attached to the lining and the bottom of the helmet 900.

Then the helmet cleaning, sterilization, deodorization and drying system 1 executes step 109 of the helmet cleaning, sterilization, deodorization and drying method: via the second turbo fan module 55, blowing away any dust on the helmet 900.

The second turbo fan module 55 is a fan, which can blow away any dust on the top and the outside of the helmet 900. Via the vibration module 60, the first turbo fan module 50 and the second turbo fan module 55, the dust attached to the helmet 900 can be removed completely.

Then the helmet cleaning, sterilization, deodorization and drying system 1 executes step 110 of the helmet cleaning, sterilization, deodorization and drying method: via the odor sensor 74, determining if the helmet 900 has an odor.

The odor sensor 74 is a common odor detector of the prior art and can determine if the helmet 900 has an odor and send the determined result to the fragrancy controller 73 to determine the dose of fragrance.

Then the helmet cleaning, sterilization, deodorization and drying system 1 executes step 111 of the helmet cleaning, sterilization, deodorization and drying method: via the fragrancy selecting unit 72, receiving the fragrance selection order.

The user can operate the button of the fragrancy selecting unit 72 to select the user's favorite fragrance. According to the button pressed by the user, the fragrancy selecting unit 72 receives the corresponding fragrance selection order and sends the fragrance selection order to the fragrancy controller 73.

Then the helmet cleaning, sterilization, deodorization and drying system 1 executes step 112 of the helmet cleaning, sterilization, deodorization and drying method: via the fragrancy controller 73, releasing one of the fragrances 71 into the helmet cleaning and sterilization compartment 12 according to the fragrance selection order.

The fragrancy controller 73 releases one of the fragrances 71 into the helmet cleaning and sterilization compartment 12 according to the fragrance selection order. It is to be known that the fragrance module 70 can only have a fragrance 71 to spray only one fragrance stably, and the fragrance module 70 can also omit the odor sensor 74 and be designed to spray a stable amount.

Then the helmet cleaning, sterilization, deodorization and drying system 1 executes step 113 of the helmet cleaning, sterilization, deodorization and drying method: via the warm air generator 32, heating the air.

As shown in FIG. 2 and FIG. 6, the warm air generator 32 is a heater, which heats up the air 903 in the first space 13.

Then the helmet cleaning, sterilization, deodorization and drying system 1 executes step 114 of the helmet cleaning, sterilization, deodorization and drying method: via the first exhaust module 37, drawing the air 903 to form the warm air 904 and causing the warm air 904 to enter the helmet cleaning and sterilization compartment 12 via the gas conduit 15 and the ventilation hole 123 to dry the helmet 900.

The first exhaust module 37 draws the air 903 which is heated to form a warm air 904 and causes the warm air 904 to enter the helmet cleaning and sterilization compartment 12 via the gas conduit 15 and the ventilation hole 123 to dry the helmet 900, thereby solving the problem of the lining of the helmet 900 absorbing moisture.

Then the helmet cleaning, sterilization, deodorization and drying system 1 executes step 115 of the helmet cleaning, sterilization, deodorization and drying method: via the humidity sensor 36, sensing the humidity of the helmet 900.

The humidity sensor 36 is a hygrometer of the prior art, which senses the humidity of the helmet 900 and sends the result to the first exhaust module 37. If the humidity sensor 36 senses that the helmet 900 is still wet, the first exhaust module 37, which receives the result, will extend the time for which warm air 904 is delivered to the helmet cleaning and sterilization compartment 12 to ensure that the helmet 900 will be fully dried.

Finally, the helmet cleaning, sterilization, deodorization and drying system 1 executes step 116 of the helmet cleaning, sterilization, deodorization and drying method: via the second exhaust module 38, extracting the air from the helmet cleaning and sterilization compartment 12 and venting it to the outside.

The second exhaust module 38 extracts the air from the helmet cleaning and sterilization compartment 12 and vents it to the outside such that the inside of the helmet cleaning and sterilization compartment 12 is clean and odorless, and another user may use the helmet cleaning, sterilization, deodorization and drying system 1 to clean a helmet 900.

Via the helmet cleaning, sterilization, deodorization and drying system and method of the present invention, the user can quickly clean, sterilize, deodorize, remove the dust from, and dry the lining of the helmet 900, and the user can add an aroma to the helmet 900 according to the user's preference to furthermore increase the comfort of wearing the helmet 900.

It is to be known that some of the abovementioned steps can be removed, adjusted in order, or repeated; for example, more important steps such as steps 104-106 and steps 113-114 can be performed first.

What is claimed is:

1. A helmet cleaning, sterilization, deodorization and drying system, used for cleaning a helmet with a antibacterial fog cleaning liquid, the helmet cleaning, sterilization, deodorization and drying system comprising:
a main body;
an ultraviolet lamp module;
a fog generator;
a warm air generator;
a first exhaust module;
a controlling system module, electrically connected to the ultraviolet lamp module, the fog generator, the warm air generator and the first exhaust module;
a helmet cleaning and sterilization compartment, located in the main body and comprising a storage space, a plate and at least one ventilation hole, wherein the storage space is used for placing the helmet, the plate is located on a bottom of the storage space, and the at least one ventilation hole is located on the plate;
a containing space, located in the main body;
a cleaning liquid tank, used for containing the antibacterial fog cleaning liquid; and
a gas conduit, located in the containing space and linked to the helmet cleaning and sterilization compartment;
via the abovementioned structures, the antibacterial fog cleaning liquid is formed into a cleaning, sterilizing and deodorizing fog via the fog generator; the cleaning, sterilizing and deodorizing fog enters the helmet cleaning and sterilization compartment via the gas conduit and the at least one ventilation hole; the warm air generator heats an air such that the first exhaust module produces a warm air; the warm air enters the helmet cleaning and sterilization compartment via the gas conduit and the at least one ventilation hole.

2. The helmet cleaning, sterilization, deodorization and drying system as claimed in claim 1, further comprising an ozone generator, wherein the ozone generator is electrically connected to the controlling system module, and the ozone generator is linked to the helmet cleaning and sterilization compartment.

3. The helmet cleaning, sterilization, deodorization and drying system as claimed in claim 1, further comprising a fragrance module, and the fragrance module is electrically connected to the controlling system module and used for producing an aroma.

4. The helmet cleaning, sterilization, deodorization and drying system as claimed in claim 3, wherein the fragrance module comprises a plurality of fragrances and a fragrancy selecting unit for allowing a user to select an aroma.

5. The helmet cleaning, sterilization, deodorization and drying system as claimed in claim 3, further comprising a first turbo fan module, wherein the first turbo fan module is located under the plate and electrically connected to the controlling system module; the first turbo fan module comprises a conduit, and the conduit is linked to the at least one ventilation hole.

6. The helmet cleaning, sterilization, deodorization and drying system as claimed in claim 1, further comprising a second turbo fan module, wherein the second turbo fan module is located at a top of the helmet cleaning and sterilization compartment and electrically connected to the controlling system module.

7. The helmet cleaning, sterilization, deodorization and drying system as claimed in claim 1, further comprising a liquid conduit and a liquid extraction module, wherein the liquid extraction module extracts the antibacterial fog cleaning liquid to the fog generator via the liquid conduit to form the cleaning, sterilizing and deodorizing fog.

8. The helmet cleaning, sterilization, deodorization and drying system as claimed in claim 1, wherein a composition of the antibacterial fog cleaning liquid comprises water and a smoking agent.

9. The helmet cleaning, sterilization, deodorization and drying system as claimed in claim 1, further comprising a humidity sensor, wherein the humidity sensor is electrically connected to the controlling system module, and the humidity sensor is located in the helmet cleaning and sterilization compartment for sensing a humidity of the helmet.

10. The helmet cleaning, sterilization, deodorization and drying system as claimed in claim 1, further comprising a second exhaust module, wherein the second exhaust module is electrically connected to the controlling system module, located in the helmet cleaning and sterilization compartment, and used for extracting the air in the helmet cleaning and sterilization compartment and venting the air to an outside.

11. A helmet cleaning, sterilization, deodorization and drying method, applied to a helmet cleaning, sterilization, deodorization and drying system for cleaning a helmet via an antibacterial fog cleaning liquid, wherein the helmet cleaning, sterilization, deodorization and drying system comprises an ultraviolet lamp module, a fog generator, a warm air generator, a first exhaust module, a controlling system module, a helmet cleaning and sterilization compartment, a cleaning liquid tank and a gas conduit; the controlling system module is electrically connected to the at least one ultraviolet lamp module, the fog generator, the warm air generator and the first exhaust module, the helmet cleaning and sterilization compartment is used for placing the helmet and comprising at least one ventilation hole, and the cleaning liquid tank is used for containing the antibacterial fog cleaning liquid, the method comprising:
via the ultraviolet lamp module, irradiating the helmet with ultraviolet light for sterilization;
starting the fog generator to heat up the fog generator;
via the fog generator, heating the antibacterial fog cleaning liquid to form a cleaning, sterilizing and deodorizing fog;
via the first exhaust module, causing the cleaning, sterilizing and deodorizing fog to enter the helmet cleaning and sterilization compartment via the gas conduit and the at least one ventilation hole to clean the helmet;
via the warm air generator, heating an air; and
via the first exhaust module, extracting the air to form a warm air, and causing the warm air to enter the helmet cleaning and sterilization compartment via the gas conduit and the at least one ventilation hole to dry the helmet.

12. The helmet cleaning, sterilization, deodorization and drying method as claimed in claim 11, wherein the helmet cleaning, sterilization, deodorization and drying system further comprises an ozone generator, the ozone generator is electrically connected to the controlling system module, and the ozone generator is linked to the helmet cleaning and sterilization compartment, the method further comprising:
via the ozone generator, generating ozone for the helmet cleaning and sterilization compartment.

13. The helmet cleaning, sterilization, deodorization and drying method as claimed in claim 11, wherein the helmet cleaning, sterilization, deodorization and drying system further comprises a fragrance module, and the fragrance module is electrically connected to the controlling system module and comprises a plurality of fragrances, a fragrancy selecting unit and a fragrancy controller, the method further comprising:
via the fragrancy selecting unit, receiving a fragrance selection order;
via the fragrancy controller, releasing one of the fragrances into the helmet cleaning and sterilization compartment according to the fragrance selection order.

14. The helmet cleaning, sterilization, deodorization and drying method as claimed in claim 11, wherein the helmet cleaning, sterilization, deodorization and drying system further comprises a first turbo fan module, and the first turbo fan module is located under the plate and electrically connected to the controlling system module, the method further comprising:
via the first turbo fan module, blowing away a dust under the helmet.

15. The helmet cleaning, sterilization, deodorization and drying method as claimed in claim 11, wherein the helmet cleaning, sterilization, deodorization and drying system further comprises a second turbo fan module, and the second turbo fan module is located at a top of the helmet cleaning and sterilization compartment and electrically connected to the controlling system module, the method further comprising:
via the second turbo fan module, blowing away a dust on the helmet.

16. The helmet cleaning, sterilization, deodorization and drying method as claimed in claim 11, wherein the helmet cleaning, sterilization, deodorization and drying system further comprises a liquid conduit and a liquid extraction module, the method further comprising:
via the liquid extraction module, transferring the antibacterial fog cleaning liquid to the fog generator via the liquid conduit to form the cleaning, sterilizing and deodorizing fog.

17. The helmet cleaning, sterilization, deodorization and drying method as claimed in claim 11, wherein the helmet cleaning, sterilization, deodorization and drying system further comprises a humidity sensor located in the helmet cleaning and sterilization compartment, and the humidity sensor is electrically connected to the controlling system module, the method further comprising:
via the humidity sensor, sensing a humidity of the helmet.

18. The helmet cleaning, sterilization, deodorization and drying method as claimed in claim 11, wherein the fragrance module further comprises an odor sensor, and the odor sensor is electrically connected to the fragrancy controller, the method further comprising:
via the odor sensor, determining if the helmet has an odor.

19. The helmet cleaning, sterilization, deodorization and drying method as claimed in claim 11, wherein the helmet cleaning, sterilization, deodorization and drying system further comprises a vibration module, and the vibration module is connected to a plate of the storage space and electrically connected to the controlling system module, the method further comprising:
via the vibration module, vibrating the plate such that a dust attached to the helmet on the plate will be dislodged.

20. The helmet cleaning, sterilization, deodorization and drying method as claimed in claim 11, wherein the helmet cleaning, sterilization, deodorization and drying system further comprises a second exhaust module, and the second exhaust module is electrically connected to the controlling system module and located in helmet cleaning and sterilization compartment, the method further comprising: via the second exhaust module, extracting the air from the helmet cleaning and sterilization compartment and venting the air to an outside.
